(19) United States
(12) Patent Application Publication
(10) Pub. No.: US 2025/0066313 A1
(43) Pub. Date: Feb. 27, 2025

(54) Methods of Synthesis of Chiral 3,5-Disubstituted Morpholine Compounds and Intermediates Useful Therein

(71) Applicant: BeiGene Switzerland GmbH, Basel (CH)

(72) Inventors: Yuanming Sun, Shanghai (CN); Qin Zhang, Shanghai (CN); Jianzhang Yang, Shanghai (CN); Zhongmin Xu, Shanghai (CN); Xinzhong Lai, Beijing (CN); Joerg Deerberg, Beijing (CN)

(21) Appl. No.: 18/680,922

(22) Filed: May 31, 2024

Related U.S. Application Data
(63) Continuation of application No. PCT/CN2022/136254, filed on Dec. 2, 2022.

(30) Foreign Application Priority Data
Dec. 2, 2021 (WO) PCT/CN2021/135094
Dec. 8, 2021 (WO) PCT/CN2021/136466

(51) Int. Cl. C07D 265/30; C07C 215/16

(52) U.S. Cl. CPC C07D 265/30; C07C 215/16; C07B 2200/13

(57) ABSTRACT

Provided herein are diastereomer-selective synthetic methods and intermediates for making chiral 3,5-disubstituted morpholine compounds, which are useful for the preparation of compounds useful as mitochondrial-derived activator of caspases (SMAC) mimetics for the treatment of proliferative diseases such as cancer.

### FIELD

Provided herein are synthetic methods and intermediates for the preparation of chiral 3,5-disubstituted morpholine compounds, which are useful for the preparation of compounds useful as mitochondrial-derived activator of caspases (SMAC) mimetics for the treatment of proliferative diseases such as cancer.

### BACKGROUND

The connection between abnormal protein phosphorylation and the cause or consequence of diseases has been known for over 20 years. Accordingly, protein kinases have become a very important group of drug targets. (See Cohen, Nature, 1:309-315 (2002), Gaestel et al. Curr.Med.Chem. 14: 2214-223 (2007); Grimminger et al. Nat. Rev. Drug Disc. 9(12):956-970 (2010)). Various protein kinase inhibitors have been used clinically in the treatment of a wide variety of diseases, such as cancer and chronic inflammatory diseases, including rheumatoid arthritis and psoriasis. (See Cohen, Eur. J. Biochem., 268:5001-5010 (2001); Protein Kinase Inhibitors for the Treatment of Disease: The Promise and the Problems, Handbook of Experimental Pharmacology, Springer Berlin Heidelberg, 167 (2005)).

Apoptosis plays a critical role in the development and homeostasis of cells in higher organisms and is a tightly regulated process to eliminate damaged or unwanted cells (Kerr, J. F., et al., Br J Cancer, 1972, 26, 239-257). Aberrations in the apoptotic process are implicated in many human diseases, including cancer, autoimmune diseases and inflammation (Nicholson, D. W., et al., Nature, 2000, 407, 810-816). Indeed, resistance to apoptosis is a hallmark of cancer (Hanahan, D., et al., cell 2000, 100, 57-70; Hanahan, D., et al., cell, 2011, 144, 646-674).

Apoptosis can be triggered through either the extrinsic stimulation of death receptors or the intrinsic stimuli released by mitochondria within the cell (Elmore, S., Toxicol Pathol, 2007, 35, 495-516). Inhibitors of apoptosis proteins (IAPs) are a class of pivotal negative regulators of both extrinsic and intrinsic apoptotic pathways. IAP was initially identified in baculovirus and able to inhibit apoptosis in the infected cells (Birnbaum, M. J., et al., J Virol, 1994, 68, 2521-2528). The IAPs are characterized by the presence of baculoviral IAP repeat (BIR) domains. BIR domain is approximately 70-80 amino acids in length and contains a Zn-binding motif which can facilitate protein-protein interactions involved in IAP function (Yang, Y. L., Cell Res, 2000, 10, 169-177). The human IAP family contains eight proteins: neuronal IAP (BIRC1), cellular IAP1 (cIAP1, BIRC2), cellular IAP2 (cIAP2, BIRC3), X chromosome-linked IAP (XIAP, BIRC4), survivin (BIRC5), ubiquitin-conjugating BIR domain enzyme apollon (BIRC6), melanoma IAP (ML-IAP, BIRC7), and IAP-like protein 2 (BIRC8). Among these, cIAP1, cIAP2 and XIAP play a direct role in apoptosis regulation (Salvesen, G. S., et al., Nat Rev Mol Cell Bio, 2002, 3, 401-410).

cIAP1 and cIAP2 (cIAPs) inhibit caspase-8 dependent extrinsic apoptotic pathway such as that induced by TNF-α through their ubiquitin ligase activity (Derakhshan, A., et al., Clin Cancer Res, 2017, 23, 1379-1387). Upon ligation of TNF-α to its receptor TNFR1, cIAPs, as well as tumor necrosis factor receptor type 1-associated death domain (TRADD), receptor-interacting serine/threonine kinase 1 (RIPK1) and TNF receptor-associated factors (TRAFs) are recruited to form complex I leading to activation of canonical nuclear factor-κB (NF-κB) pathway, well known to promote inflammation, proliferation and cell survival (Samuel T., et al., J Biol Chem, 2006, 281, 1080-1090; Vince J. E., et al., J Biol Chem, 2009, 284, 35906-35915; Wang C., et al., Nature, 2001, 412, 346-351).

XIAP is the only IAP protein that inhibits both extrinsic and intrinsic apoptotic pathways by directly counteracting caspase activation through their BIR domains (Deveraux Q. L., et al., Nature, 1997, 388, 300-304). The BIR2 domain and the preceding linker region of XIAP associates to the IAP-binding motif (IBM) and active site of caspase-3 and -7, the executioner caspases shared by extrinsic and intrinsic apoptosis, and inhibits their function (Chai J., et al., Cell, 2001, 104, 769-780; Riedl S. J., et al., Cell, 2001, 104, 791-800). XIAP binds to pro-caspase-9 via its BIR3 domain and prevents the dimerization and subsequent activation of caspase-9, the critical initiator caspase in the intrinsic pathway (Shiozaki E. N., et al., Mol Cell, 2003, 11, 519-527).

cIAP1, cIAP2 and XIAP proteins are broadly expressed in various tumor types. And positive expression of cIAPs and XIAP is associated with high-grade cancer and poor prognosis (Che X., et al., Urol Oncol, 2012, 30, 450-456; Yang C., et al., J Exp Clin Cancer Res, 2016, 35, 158). Moreover, downregulation or depletion of these IAPs has shown to restore sensitivity to extrinsic or intrinsic apoptotic stimuli (Gu H., et al., Aging(Albany NY), 2018, 10, 1597-1608). Taken together, targeting IAP proteins provides a potential anti-tumor strategy.

The second mitochondrial-derived activator of caspases (SMAC), also known as direct IAP binding protein with low pI (DIABLO), is an endogenous antagonist of cIAP1, cIAP2 and XIAP to promote apoptosis (Du C., et al., Cell, 2000, 102, 33-42; Verhagen A. M., et al., Cell, 2000, 102, 43-53). SMAC is normally sequestered in the mitochondria and released into cytosol when cells undergo apoptosis. In cytosol, the N-terminal mitochondria-targeting sequence of SMAC is cleaved to expose the tetrapeptide (Ala-Val-Pro-Ile) that allows SMAC to interact with the BIR domains of IAPs (Chai J., et al., Nature, 2000, 406, 855-862). Binding of SMAC to BIR3 domain of cIAP1 and cIAP2 stimulates their E3 ubiquitin ligase activity and induces their proteasomal degradation. Loss of cIAP proteins promotes the formation of RIPK1, caspase-8 and Fas-associated protein with death domain (FADD) containing complex II and triggers TNF-α mediated apoptosis (Dueber E. C., et al., Science, 2011, 334, 376-380). Dimerized SMAC binds to the BIR2 and BIR3 domains of XIAP and disrupts its interaction with caspase-3, -7 and -9, leading to caspase-dependent apoptosis (Micheau, O., et al., Cell, 2003, 114, 181-190; Chai J., et al., Cell, 2001, 104, 769-780; Liu Z., et al., Nature, 2000, 408, 1004-1008).

SMAC mimetics are small molecules that contain 4 amino acids that mimic the N-terminal (Ala-Val-Pro-Ile) of SMAC. Similar to SMAC, SMAC mimetics bind to BIR domains of IAPs and antagonize their function to promote apoptosis in cancer cells (Chai J., et al., Cell, 2001, 104, 769-780; Dueber E. C., et al., Science, 2011, 334, 376-380; Liu Z., et al., Nature, 2000, 408, 1004-1008; Verhagen A. M., et al., Cell, 2000, 102, 43-53). Taken together, SMAC mimetics become a new class of cancer therapeutic candidates.

PCT/CN2021/098123 filed on Jun. 3, 2021 teaches a group of compounds as the SMAC mimetics and further teaches that a R,R-3,5-dimethyl-morpholine moiety is needed in most of the SMAC mimetics in the application. However, a stereoselective synthetic method does not appear to be available in the art for the preparation of R,R-3,5-dimethyl-morpholine intermediates useful for the preparation of the SMAC mimetics.

Dieter Enders, et al., Synthesis, 1994(01), 66-72, reported a diastereo-random synthetic route of 3,5-dimethylmorpholine, producing an equimolar mixture of (S,S)- and meso-bis(beta-hydroxyisopropyl)amine as a synthetic intermediate. G. Cignarella et al., Gazz. Chim. Ital., 1962, 92, 3-16, reported 2,2′-(benzylazanediyl)bis(propan-1-ol) as an oil with boiling point of 133-135° C. at 0.2 mm Hg and a diastereo-random synthetic route of 2,2′-(benzylazanediyl)bis(propan-1-ol). L. Fontanella et al., Il Farmaco, Ed. Sci., 1982, 37(6), 378-386, reported another diastereo-random synthetic route of 2,2′-(benzylazanediyl)bis(propan-1-01).

Thus, for purposes of pharmaceutical manufacture, particularly control of drug quality and cost, there is a need to develop a stereoselective synthetic method to prepare intermediates useful for the preparation of certain SMAC mimetics.

Citation or identification of any reference in this section is not to be construed as an admission that the reference is prior art to the present application.

### SUMMARY

Provided herein are diastereo-selective methods for preparing a compound of Formula (VIII):

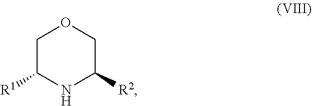

pharmaceutically acceptable salts, solid forms, enantiomer, isotopologues, and solvates thereof,
wherein the method comprises contacting a compound of Formula (VII),

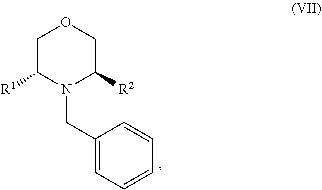

with hydrogen (H₂) in the presence of a catalyst in a solvent.
wherein R¹, and R² are as provided herein.

In one embodiment, the compound of Formula (VII) is prepared by contacting a compound of Formula (VI),

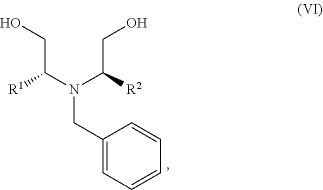

with an acid.

In one embodiment, the compound of Formula (VI) is prepared by contacting a mixture of a compound of Formula (IV),

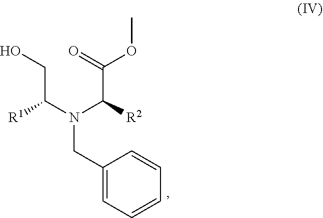

and
a compound of Formula (V),

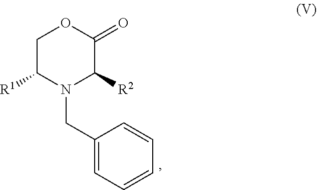

with a reducing agent.

In one embodiment, the mixture of a compound of Formula (IV) and a compound of Formula (V) is prepared by contacting a compound of Formula (II),

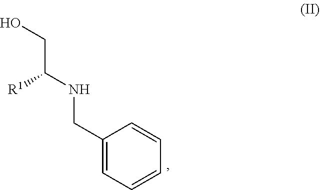

with a compound of Formula (III)

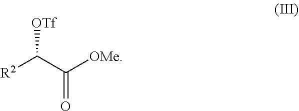

In one embodiment, the compound of Formula (II) is prepared by contacting a compound of Formula (I),

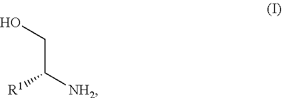

with PhCHO.

Provided herein are compounds of Formula (VI),

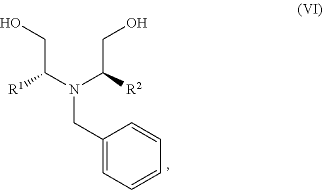

pharmaceutically acceptable salts, solid forms, enantiomer, isotopologues, and solvates thereof,
wherein R¹, and R² are as provided herein.

Provided herein is Compound 6 having the name of (2R,2′R)-2,2′-(benzylazanediyl)bis(propan-1-ol) with the following structure:

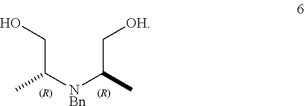

In one embodiment, Compound 6 is a crystalline form which has an X-ray powder diffraction pattern comprising peaks at approximately 22.54, 27.09, and 27.30° 2θ.

Provided herein are processes and intermediates useful for the preparation of SMAC mimetics disclosed in PCT/CN2021/098123 filed on Jun. 3, 2021.

The present embodiments can be understood more fully by reference to the detailed description and examples, which are intended to exemplify non-limiting embodiments.

### BRIEF DESCRIPTION OF THE DRAWINGS

FIG. 1 depicts a ¹H nuclear magnetic resonance (NMR) spectrum of Compound 2.

FIG. 2 depicts a ¹H NMR spectrum of Compound 6.

FIG. 3 depicts a ¹H NMR spectrum of Compound 7.

FIG. 4 depicts a ¹H NMR spectrum of Compound 8.

FIG. 5 depicts an X-ray powder diffractogram (XRPD) pattern of Compound 6.

FIG. 6 depicts a differential scanning calorimetry (DSC) thermogram of Compound 6.

FIG. 7 depicts a thermogravimetric analysis (TGA) thermogram of Compound 6.

### DETAILED DESCRIPTION

### Definitions

As used herein, and in the specification and the accompanying claims, the indefinite articles “a” and “an” and the definite article “the” include plural as well as single referents, unless the context clearly indicates otherwise.

As used herein, and unless otherwise specified, the terms “about” and “approximately,” when used in connection with amounts, or weight percentage of ingredients of a composition, mean an amount, or weight percent that is recognized by one of ordinary skill in the art to provide a pharmacological effect equivalent to that obtained from the specified amount, or weight percent. In certain embodiments, the terms “about” and “approximately,” when used in this context, contemplate an amount, or weight percent within 30%, within 20%, within 15%, within 10%, or within 5%, of the specified amount, or weight percent.

As used herein, and unless otherwise specified, the terms “about” and “approximately,” when used in connection with a numeric value or range of values which is provided to characterize a particular solid form, e.g., a specific temperature or temperature range, such as, for example, that describes a melting, dehydration, desolvation, or glass transition temperature; a mass change, such as, for example, a mass change as a function of temperature or humidity; a solvent or water content, in terms of, for example, mass or a percentage; or a peak position, such as, for example, in analysis by, for example, IR or Raman spectroscopy or XRPD; indicate that the value or range of values may deviate to an extent deemed reasonable to one of ordinary skill in the art while still describing the solid form. Techniques for characterizing crystal forms and amorphous solids include, but are not limited to, thermal gravimetric analysis (TGA), differential scanning calorimetry (DSC), X-ray powder diffractometry (XRPD), single-crystal X-ray diffractometry, vibrational spectroscopy, e.g., infrared (IR) and Raman spectroscopy, solid-state and solution nuclear magnetic resonance (NMR) spectroscopy, optical microscopy, hot stage optical microscopy, scanning electron microscopy (SEM), electron crystallography and quantitative analysis, particle size analysis (PSA), surface area analysis, solubility studies, and dissolution studies. In certain embodiments, the terms “about” and “approximately,” when used in this context, indicate that the numeric value or range of values may vary within 30%, 20%, 15%, 10%, 9%, 8%, 7%, 6%, 5%, 4%, 3%, 2%, 1.5%, 1%, 0.5%, or 0.25% of the recited value or range of values. For example, in some embodiments, the value of an XRPD peak position may vary by up to ±0.2° 2θ (or ±0.2 degree 2θ) while still describing the particular XRPD peak.

As used herein, and unless otherwise specified, a crystalline compound that is “pure,” i.e., substantially free of other crystalline or amorphous solids, contains less than about 10% by weight of one or more other crystalline or amorphous solids, less than about 5% by weight of one or more other crystalline or amorphous solids, less than about 3% by weight of one or more other crystalline or amorphous solids, or less than about 1% by weight of one or more other crystalline or amorphous solids.

As used herein, and unless otherwise specified, a solid form that is “substantially physically pure” is substantially free from other solid forms. In certain embodiments, a crystal form that is substantially physically pure contains less than about 10%, 9%, 8%, 7%, 6%, 5%, 4%, 3%, 2%, 1%, 0.5%, 0.4%, 0.3%, 0.2%, 0.1%, 0.05%, or 0.01% of one or more other solid forms on a weight basis. The detection of other solid forms can be accomplished by any method apparent to a person of ordinary skill in the art, including, but not limited to, diffraction analysis, thermal analysis, elemental combustion analysis and/or spectroscopic analysis.

As used herein, and unless otherwise specified, a solid form that is “substantially chemically pure” is substantially free from other chemical compounds (i.e., chemical impurities). In certain embodiments, a solid form that is substantially chemically pure contains less than about 10%, 9%, 8%, 7%, 6%, 5%, 4%, 3%, 2%, %, 0.5%, 0.4%, 0.3%, 0.2%, 0.1%, 0.05%, or 0.01% of one or more other chemical compounds on a weight basis. The detection of other chemical compounds can be accomplished by any method apparent to a person of ordinary skill in the art, including, but not limited to, methods of chemical analysis, such as, e.g., mass spectrometry analysis, spectroscopic analysis, thermal analysis, elemental combustion analysis and/or chromatographic analysis.

As used herein, and unless otherwise indicated, a chemical compound, solid form, or composition that is “substantially free” of another chemical compound, solid form, or composition means that the compound, solid form, or composition contains, in certain embodiments, less than about 10%, 9%, 8%, 7%, 6%, 5%, 4%, 3%, 2%, 1%, 0.5%, 0.4%, 0.3%, 0.2% 0.1%, 0.05%, or 0.01% by weight of the other compound, solid form, or composition.

An “alkyl” group is a saturated, partially saturated, or unsaturated straight chain or branched non-cyclic hydrocarbon having from 1 to 10 carbon atoms, typically from 1 to 8 carbons or, in some embodiments, from 1 to 6, 1 to 4, or 2 to 6 or 2 to 4 carbon atoms. Representative alkyl groups include -methyl, -ethyl, -n-propyl, -n-butyl, -n-pentyl, and -n-hexyl; while saturated branched alkyls include -isopropyl, -sec-butyl, -isobutyl, -tert-butyl, -isopentyl, -neopentyl, -tert-pentyl, -2-methylphenyl, -3-methylphenyl, -4-methylphenyl, -2,3-dimethylbutyl and the like. Examples of unsaturated alkyl groups include, but are not limited to, vinyl, allyl, —CH═CH(CH₃), —CH═C(CH₃)₂, —C(CH₃)═CH₂, —C(CH₃)═CH(CH₃), —C(CH₂CH₃)═CH₂, —C≡CH, —C≡C(CH₃), —C≡C(CH₂CH₃), —CH₂C≡CH, —CH₂C≡C(CH₃) and —CH₂C≡C(CH₂CH₃), among others. An alkyl group can be substituted or unsubstituted. When the alkyl groups described herein are said to be “substituted,” they may be substituted with any substituent or substituents as those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; B(OH)₂, or O(alkyl)aminocarbonyl.

A “cycloalkyl” group is a saturated, or partially saturated cyclic alkyl group of from 3 to 10 carbon atoms having a single cyclic ring or multiple condensed or bridged rings which can be optionally substituted with from 1 to 3 alkyl groups. In some embodiments, the cycloalkyl group has 3 to 8 ring members, whereas in other embodiments the number of ring carbon atoms ranges from 3 to 5, 3 to 6, or 3 to 7. Such cycloalkyl groups include, by way of example, single ring structures such as cyclopropyl, cyclobutyl, cyclopentyl, cyclohexyl, cycloheptyl, cyclooctyl, 1-methylcyclopropyl, 2-methylcyclopentyl, 2-methylcyclooctyl, and the like, or multiple or bridged ring structures such as 1-bicyclo[1.1.1]pentyl, bicyclo[2.1.1]hexyl, bicyclo[2.2.1]heptyl, bicyclo[2.2.2]octyl, adamantyl and the like. Examples of unsaturated cycloalkyl groups include cyclohexenyl, cyclopentenyl, cyclohexadienyl, butadienyl, pentadienyl, hexadienyl, among others. A cycloalkyl group can be substituted or unsubstituted. Such substituted cycloalkyl groups include, by way of example, cyclohexanol and the like.

An “aryl” group is an aromatic carbocyclic group of from 6 to 14 carbon atoms having a single ring (e.g., phenyl) or multiple condensed rings (e.g., naphthyl or anthryl). In some embodiments, aryl groups contain 6-14 carbons, and in others from 6 to 12 or even 6 to 10 carbon atoms in the ring portions of the groups. Particular aryls include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

A “heteroaryl” group is an aryl ring system having one to four heteroatoms as ring atoms in a heteroaromatic ring system, wherein the remainder of the atoms are carbon atoms. In some embodiments, heteroaryl groups contain 3 to 6 ring atoms, and in others from 6 to 9 or even 6 to 10 atoms in the ring portions of the groups. Suitable heteroatoms include oxygen, sulfur and nitrogen. In certain embodiments, the heteroaryl ring system is monocyclic or bicyclic. Non-limiting examples include but are not limited to, groups such as pyrrolyl, pyrazolyl, imidazolyl, triazolyl, tetrazolyl, oxazolyl, isoxazolyl, benzisoxazolyl (e.g., benzo[d]isoxazolyl), thiazolyl, pyrolyl, pyridazinyl, pyrimidyl, pyrazinyl, thiophenyl, benzothiophenyl, furanyl, benzofuranyl, indolyl (e.g., indolyl-2-onyl or isoindolin-1-onyl), azaindolyl (pyrrolopyridyl or 1H-pyrrolo[2,3-b]pyridyl), indazolyl, benzimidazolyl (e.g., 1H-benzo[d]imidazolyl), imidazopyridyl (e.g., azabenzimidazolyl or 1H-imidazo[4,5-b]pyridyl), pyrazolopyridyl, triazolopyridyl, benzotriazolyl (e.g., 1H-benzo[d][1,2,3]triazolyl), benzoxazolyl (e.g., benzo[d]oxazolyl), benzothiazolyl, benzothiadiazolyl, isoxazolopyridyl, thianaphthalenyl, purinyl, xanthinyl, adeninyl, guaninyl, quinolinyl, isoquinolinyl (e.g., 3,4-dihydroisoquinolin-1(2H)-onyl), tetrahydroquinolinyl, quinoxalinyl, and quinazolinyl groups.

A “heterocyclyl” is an aromatic (also referred to as heteroaryl) or non-aromatic cycloalkyl in which one to four of the ring carbon atoms are independently replaced with a heteroatom from the group consisting of O, S and N. In some embodiments, heterocyclyl groups include 3 to 10 ring members, whereas other such groups have 3 to 5, 3 to 6, or 3 to 8 ring members. Heterocyclyls can also be bonded to other groups at any ring atom (i.e., at any carbon atom or heteroatom of the heterocyclic ring). A heterocycloalkyl group can be substituted or unsubstituted. Heterocyclyl groups encompass unsaturated, partially saturated and saturated ring systems, such as, for example, imidazolyl, imidazolinyl and imidazolidinyl (e.g., imidazolidin-4-one or imidazolidin-2,4-dionyl) groups. The phrase heterocyclyl includes fused ring species, including those comprising fused aromatic and non-aromatic groups, such as, for example, 1- and 2-aminotetraline, benzotriazolyl (e.g., 1H-benzo[d][1,2,3]triazolyl), benzimidazolyl (e.g., 1H-benzo[d]imidazolyl), 2,3-dihydrobenzo[1,4]dioxinyl, and benzo[1,3]dioxolyl. The phrase also includes bridged polycyclic ring systems containing a heteroatom such as, but not limited to, quinuclidyl. Representative examples of a heterocyclyl group include, but are not limited to, aziridinyl, azetidinyl, azepanyl, oxetanyl, pyrrolidyl, imidazolidinyl (e.g., imidazolidin-4-onyl or imidazolidin-2,4-dionyl), pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, benzisoxazolyl (e.g., benzo[d]isoxazolyl), thiazolyl, thiazolinyl, isothiazolyl, thiadiazolyl, oxadiazolyl, piperidyl, piperazinyl (e.g., piperazin-2-onyl), morpholinyl, thiomorpholinyl, tetrahydropyranyl (e.g., tetrahydro-2H-pyranyl), tetrahydrothiopyranyl, oxathianyl, dioxyl, dithianyl, pyranyl, pyridyl, pyrimidyl, pyridazinyl, pyrazinyl, triazinyl, dihydropyridyl, dihydrodithiinyl, dihydrodithionyl, 1,4-dioxaspiro[4.5]decanyl, homopiperazinyl, quinuclidyl, indolyl (e.g., indolyl-2-onyl or isoindolin-1-onyl), indolinyl, isoindolyl, isoindolinyl, azaindolyl (pyrrolopyridyl or 1H-pyrrolo[2,3-b]pyridyl), indazolyl, indolizinyl, benzotriazolyl (e.g. 1H-benzo[d][1,2,3]triazolyl), benzimidazolyl (e.g., 1H-benzo[d]imidazolyl or 1H-benzo[d]imidazol-2(3H)-onyl), benzofuranyl, benzothiophenyl, benzothiazolyl, benzoxadiazolyl, benzoxazinyl, benzodithiinyl, benzoxathiinyl, benzothiazinyl, benzoxazolyl (i.e., benzo[d]oxazolyl), benzothiazolyl, benzothiadiazolyl, benzo[1,3]dioxolyl, pyrazolopyridyl (for example, 1H-pyrazolo[3,4-b]pyridyl, 1H-pyrazolo[4,3-b]pyridyl), imidazopyridyl (e.g., azabenzimidazolyl or 1H-imidazo[4,5-b]pyridyl), triazolopyridyl, isoxazolopyridyl, purinyl, xanthinyl, adeninyl, guaninyl, quinolinyl, isoquinolinyl (e.g., 3,4-dihydroisoquinolin-1(2H)-onyl), quinolizinyl, quinoxalinyl, quinazolinyl, cinnolinyl, phthalazinyl, naphthyridinyl, pteridinyl, thianaphthalenyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, tetrahydropyrimidin-2(1H)-one and tetrahydroquinolinyl groups. Representative non-aromatic heterocyclyl groups do not include fused ring species that comprise a fused aromatic group. Examples of non-aromatic heterocyclyl groups include aziridinyl, azetidinyl, azepanyl, pyrrolidyl, imidazolidinyl (e.g., imidazolidin-4-onyl or imidazolidin-2,4-dionyl), pyrazolidinyl, thiazolidinyl, tetrahydrothiophenyl, tetrahydrofuranyl, piperidyl, piperazinyl (e.g., piperazin-2-onyl), morpholinyl, thiomorpholinyl, tetrahydropyranyl (e.g., tetrahydro-2H-pyranyl), tetrahydrothiopyranyl, oxathianyl, dithianyl, 1,4-dioxaspiro[4.5]decanyl, homopiperazinyl, quinuclidyl, or tetrahydropyrimidin-2(1H)-one. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed below.

A “cycloalkylalkyl” group is a radical of the formula: -alkyl-cycloalkyl, wherein alkyl and cycloalkyl are as defined above. Substituted cycloalkylalkyl groups may be substituted at the alkyl, the cycloalkyl, or both the alkyl and the cycloalkyl portions of the group. Representative cycloalkylalkyl groups include but are not limited to methylcyclopropyl, methylcyclobutyl, methylcyclopentyl, methylcyclohexyl, ethylcyclopropyl, ethylcyclobutyl, ethylcyclopentyl, ethylcyclohexyl, propylcyclopentyl, propylcyclohexyl and the like.

An “aralkyl” group is a radical of the formula: -alkyl-aryl, wherein alkyl and aryl are defined above. Substituted aralkyl groups may be substituted at the alkyl, the aryl, or both the alkyl and the aryl portions of the group. Representative aralkyl groups include but are not limited to benzyl and phenethyl groups and fused (cycloalkylaryl)alkyl groups such as 4-ethyl-indanyl.

An “heterocyclylalkyl” group is a radical of the formula: -alkyl-heterocyclyl, wherein alkyl and heterocyclyl are defined above. Substituted heterocyclylalkyl groups may be substituted at the alkyl, the heterocyclyl, or both the alkyl and the heterocyclyl portions of the group. Representative heterocylylalkyl groups include but are not limited to 4-ethyl-morpholinyl, 4-propylmorpholinyl, furan-2-yl methyl, furan-3-yl methyl, pyridin-3-yl methyl, tetrahydrofuran-2-yl ethyl, and indol-2-yl propyl. When the groups described herein, with the exception of alkyl group, are said to be “substituted,” they may be substituted with any appropriate substituent or substituents. Illustrative examples of substituents are those found in the exemplary compounds and embodiments disclosed herein, as well as halogen (chloro, iodo, bromo, or fluoro); alkyl; hydroxyl; alkoxy; alkoxyalkyl; amine; alkylamine; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; oxygen (0); B(OH)₂, O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, or thiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazinyl, pyridazinyl, pyrimidyl, benzimidazolyl, benzothiophenyl, or benzofuranyl) aryloxy; aralkyloxy; heterocyclyloxy; and heterocyclyl alkoxy.

A “halogen” is chloro, iodo, bromo, or fluoro.

A “hydroxyalkyl” group is an alkyl group as described above substituted with one or more hydroxy groups.

An “alkoxy” group is —O-(alkyl), wherein alkyl is defined above.

An “alkoxyalkyl” group is -(alkyl)-O-(alkyl), wherein alkyl is defined above.

An “amine” group is a radical of the formula: —NH₂.

A “hydroxyl amine” group is a radical of the formula: —N(R^{#})OH or —NHOH, wherein R^{#} is a substituted or unsubstituted alkyl, cycloalkyl, cycloalkylalkyl, aryl, aralkyl, heterocyclyl or heterocyclylalkyl group as defined herein.

An “alkoxyamine” group is a radical of the formula: —N(R^{#})O-alkyl or —NHO-alkyl, wherein R^{#} is as defined above.

An “aralkoxyamine” group is a radical of the formula: —N(R^{#})O-aryl or —NHO-aryl, wherein R^{#} is as defined above.

An “alkylamine” group is a radical of the formula: —NH-alkyl or —N(alkyl)₂, wherein each alkyl is independently as defined above.

An “aminocarbonyl” group is a radical of the formula: —C(═O)N(R^{#})₂, —C(═O)NH(R^{#}) or —C(═O)NH₂, wherein each R^{#} is as defined above.

An “acylamino” group is a radical of the formula: —NHC(═O)(R^{#}) or —N(alkyl)C(═O)(R^{#}), wherein each alkyl and R^{#} are independently as defined above.

An “O(alkyl)aminocarbonyl” group is a radical of the formula: —O(alkyl)C(═O)N(R^{#})₂, —O(alkyl)C(═O)NH(R^{#}) or —O(alkyl)C(═O)NH₂, wherein each R^{#} is independently as defined above.

An “N-oxide” group is a radical of the formula: —N⁺—O—.

A “carboxy” group is a radical of the formula: —C(═O)OH.

A “ketone” group is a radical of the formula: —C(═O)(R^{#}), wherein R^{#} is as defined above.

An “aldehyde” group is a radical of the formula: —CH(═O).

An “ester” group is a radical of the formula: —C(═O)O(R^{#}) or —OC(═O)(R^{#}), wherein R^{#} is as defined above.

A “urea” group is a radical of the formula: —N(alkyl)C(═O)N(R^{#})₂, N(alkyl)C(═O)NH(R^{#}), —N(alkyl)C(═O)NH₂, —NHC(═O)N(R^{#})₂, —NHC(═O)NH(R^{#}), or —NHC(═O)NH₂^{#}, wherein each alkyl and R^{#} are independently as defined above.

An “imine” group is a radical of the formula: —N═C(R^{#})₂ or —C(R^{#})═N(R^{#}), wherein each R^{#} is independently as defined above.

An “imide” group is a radical of the formula: —C(═O)N(R^{#})C(═O)(R^{#}) or —N((C═O)(R^{#}))₂, wherein each R^{#} is independently as defined above.

A “urethane” group is a radical of the formula: —OC(═O)N(R^{#})₂, —OC(═O)NH(R^{#}), —N(R^{#})C(═O)O(R^{#}), or —NHC(═O)O(R^{#}), wherein each R^{#} is independently as defined above.

An “amidine” group is a radical of the formula: —C(═N(R^{#}))N(R^{#})₂, —C(═N(R^{#}))NH(R^{#}), —C(═N(R^{#}))NH₂, —C(═NH)N(R^{#})₂, —C(═NH)NH(R^{#}), —C(═NH)NH₂, —N═C(R^{#})N(R^{#})₂, —N═C(R^{#})NH(R^{#}), —N═C(R^{#}) NH₂, —N(R^{#})C(R^{#})═N(R^{#}), —NHC(R^{#})═N(R^{#}), —N(R^{#})C(R^{#})═NH, or —NHC(R^{#})═NH, wherein each R^{#} is independently as defined above.

A “guanidine” group is a radical of the formula: —N(R^{#})C(═N(R⁴))N(R⁴)₂, —NHC(═N(R^{#}))N(R^{#})₂, —N(R^{#})C(═NH)N(R^{#})₂, —N(R^{#})C(═N(R^{#}))NH(R^{#}), —N(R^{#})C(═N(R^{#}))NH₂, —NHC(═NH)N(R^{#})₂, —NHC(═N(R^{#}))NH(R^{#}), —NHC(═N(R^{#}))NH₂, —NHC(═NH)NH(R^{#}), —NHC(═NH)NH₂, —N═C(N(R^{#})₂)₂, —N═C(NH(R^{#}))₂, or —N═C(NH₂)₂, wherein each R^{#} is independently as defined above.

A “enamine” group is a radical of the formula: —N(R^{#})C(R^{#})═C(R^{#})₂, —NHC(R^{#})═C(R^{#})₂, —C(N(R^{#})₂)═C(R^{#})₂, —C(NH(R^{#}))═C(R^{#})₂, —C(NH₂)═C(R^{#})₂, —C(R^{#})═C(R^{#})(N(R^{#})₂), —C(R^{#})═C(R^{#})(NH(R^{#})) or —C(R^{#})═C(R^{#})(NH₂), wherein each R^{#} is independently as defined above.

An “oxime” group is a radical of the formula: —C(═NO(R^{#}))(R^{#}), —C(═NOH)(R^{#}), —CH(═NO(R^{h})), or —CH(═NOH), wherein each R^{#} is independently as defined above.

A “hydrazide” group is a radical of the formula: —C(═O)N(R^{#})N(R^{#})₂, —C(═O)NH(R^{#})₂, —C(═O)N(R^{#})NH(R^{#}), —C(═O)N(R^{#})NH₂, —C(═O)NHNH(R^{#})₂, or —C(═O)NHNH₂, wherein each R^{#} is independently as defined above.

A “hydrazine” group is a radical of the formula: —N(R^{#})N(R^{#})₂, —NHN(R^{#})₂, —N(R^{#})NH(R^{#}), —N(R^{#})NH₂, —NHNH(R^{#})₂, or —NHNH₂, wherein each R^{#} independently as defined above.

A “hydrazone” group is a radical of the formula: —C(═N—N(R^{#})₂)(R^{#})₂, —C(═N—NH(R^{#}))(R^{#})₂, —C(═N—NH₂)(R^{#})₂, —N(R^{#})(N═C(R^{#})₂), or —NH(N═C(R^{#})₂), wherein each R^{#} independently as defined above.

An “azide” group is a radical of the formula: —N₃.

An “isocyanate” group is a radical of the formula: —N═C═O.

An “isothiocyanate” group is a radical of the formula: —N═C═S.

A “cyanate” group is a radical of the formula: —OCN.

A “thiocyanate” group is a radical of the formula: —SCN.

A “thioether” group is a radical of the formula; —S(R^{#}), wherein R^{#} as defined above.

A “thiocarbonyl” group is a radical of the formula: —C(═S)(R^{#}), wherein R^{#} as defined above.

A “sulfinyl” group is a radical of the formula: —S(═O)(R^{#}), wherein R^{#} as defined above.

A “sulfone” group is a radical of the formula: —S(═O)₂(R^{#}), wherein R^{#} as defined above.

A “sulfonylamino” group is a radical of the formula: —NHSO₂(R^{#}) or —N(alkyl)SO₂(R^{#}), wherein each alkyl and R are defined above.

A “sulfonamide” group is a radical of the formula: —S(═O)₂N(R^{#})₂, or —S(═O)₂NH(R^{#}), or —S(═O)₂NH₂, wherein each R^{#} independently as defined above.

A “phosphonate” group is a radical of the formula: —P(═O)(O(R^{#}))₂, —P(═O)(OH)₂,—OP(═O)(O(R^{#}))(R^{#}), or —OP(═O)(OH)(R^{#}), wherein each R is independently as defined above.

A “phosphine” group is a radical of the formula: —P(R^{#})₂, wherein each R^{#} is independently as defined above.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

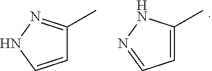

As readily understood by one skilled in the art, a wide variety of functional groups and other structures may exhibit tautomerism and all tautomers of Compound 6 are within the scope of the present invention.

Unless otherwise specified, the term “composition” as used herein is intended to encompass a product comprising the specified ingredient(s) (and in the specified amount(s), if indicated), as well as any product which results, directly or indirectly, from combination of the specified ingredient(s) in the specified amount(s). By “pharmaceutically acceptable,” it is meant a diluent, excipient, or carrier in a formulation must be compatible with the other ingredient(s) of the formulation and not deleterious to the recipient thereof.

The term “solid form” refers to a physical form which is not predominantly in a liquid or a gaseous state. As used herein and unless otherwise specified, the term “solid form,” when used herein to refer to Compound 6, refers to a physical form comprising Compound 6 which is not predominantly in a liquid or a gaseous state. A solid form may be a crystalline form or a mixture thereof. In certain embodiments, a solid form may be a liquid crystal. In certain embodiments, the term “solid forms comprising Compound 6” includes crystal forms comprising Compound 6. In certain embodiments, the solid form of Compound 6 is Form A, the amorphous solid, or a mixture thereof.

As used herein and unless otherwise specified, the term “crystalline” when used to describe a compound, substance, modification, material, component or product, unless otherwise specified, means that the compound, substance, modification, material, component or product is substantially crystalline as determined by X-ray diffraction. See, e.g., Remington: The Science and Practice of Pharmacy, 21st edition, Lippincott, Williams and Wilkins, Baltimore, MD (2005); The United States Pharmacopeia, 23^{rd} ed., 1843-1844 (1995).

The term “crystal form” or “crystalline form” refers to a solid form that is crystalline. In certain embodiments, a crystal form of a substance may be substantially free of amorphous solids and/or other crystal forms. In certain embodiments, a crystal form of a substance may contain less than about 1%, less than about 2%, less than about 3%, less than about 4%, less than about 5%, less than about 6%, less than about 7%, less than about 8%, less than about 9%, less than about 10%, less than about 15%, less than about 20%, less than about 25%, less than about 30%, less than about 35%, less than about 40%, less than about 45%, or less than about 50% by weight of one or more amorphous solids and/or other crystal forms. In certain embodiments, a crystal form of a substance may be physically and/or chemically pure. In certain embodiments, a crystal form of a substance may be about 99%, about 98%, about 97%, about 96%, about 95%, about 94%, about 93%, about 92%, about 91%, or about 90% physically and/or chemically pure.

Unless otherwise specified, the term “amorphous” or “amorphous solid” means that the substance, component, or product in question is not substantially crystalline as determined by X-ray diffraction. In particular, the term “amorphous solid” describes a disordered solid form, i.e., a solid form lacking long range crystalline order. In certain embodiments, an amorphous solid of a substance may be substantially free of other amorphous solids and/or crystal forms. In certain embodiments, an amorphous solid of a substance may contain less than about 1%, less than about 2%, less than about 3%, less than about 4%, less than about 5%, less than about 10%, less than about 15%, less than about 20%, less than about 25%, less than about 30%, less than about 35%, less than about 40%, less than about 45%, or less than about 50% by weight of one or more other amorphous solids and/or crystal forms on a weight basis. In certain embodiments, an amorphous solid of a substance may be physically and/or chemically pure. In certain embodiments, an amorphous solid of a substance be about 99%, about 98%, about 97%, about 96%, about 95%, about 94%, about 93%, about 92%, about 91%, or about 90% physically and/or chemically pure.

The term “Diastereomeric Ratio” or “dr.” refers to the ratio of the molar percentage of one diastereoisomer in a mixture to that of the other.

Unless otherwise specified, the terms “solvate” and “solvated,” as used herein, refer to a solid form of a substance which contains solvent. The terms “hydrate” and “hydrated” refer to a solvate wherein the solvent is water. “Polymorphs of solvates” refer to the existence of more than one solid form for a particular solvate composition. Similarly, “polymorphs of hydrates” refer to the existence of more than one solid form for a particular hydrate composition. The term “desolvated solvate,” as used herein, refers to a solid form of a substance which can be made by removing the solvent from a solvate. The terms “solvate” and “solvated,” as used herein, can also refer to a solvate of a salt, cocrystal, or molecular complex. The terms “hydrate” and “hydrated,” as used herein, can also refer to a hydrate of a salt, cocrystal, or molecular complex.

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable salts include, but are not limited to, those salts which are, within the scope of sound medical judgment, suitable for use in contact with the tissues of humans and lower animals without undue toxicity, irritation, allergic response, and the like, and are commensurate with a reasonable benefit/risk ratio. A pharmaceutically acceptable salt may be prepared in situ during the final isolation and purification of the compounds disclosed herein, or separately by reacting the free base function with a suitable organic acid or by reacting the acidic group with a suitable base.

It should also be noted the compounds can contain unnatural proportions of atomic isotopes at one or more of the atoms. For example, the compounds may be radiolabeled with radioactive isotopes, such as for example tritium (³H), iodine-125 (¹²⁵I), sulfur-35 (³⁵S), or carbon-14 (¹⁴C), or may be isotopically enriched, such as with deuterium (²H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer and inflammation therapeutic agents, research reagents, e.g., binding assay reagents, and diagnostic agents, e.g., in vivo imaging agents. All isotopic variations of the compounds as described herein whether radioactive or not, are intended to be encompassed within the scope of the embodiments provided herein. In some embodiments, there are provided isotopologues of the compounds, for example, the isotopologues are deuterium, carbon-13, or nitrogen-15 enriched compounds.

In addition, if the stereochemistry of a structure or a portion of a structure is not indicated with, for example, bold or dashed lines, the structure or portion of the structure is to be interpreted as encompassing all stereoisomers of it.

### Methods for Making

Provided herein are processes and intermediates useful for the preparation of SMAC mimetics disclosed in PCT/CN2021/098123 filed on Jun. 3, 2021.

By way of example and not limitation, the compound of Formula (VIII) can be prepared as outlined in Scheme 1 shown below, as well as in the examples set forth herein.

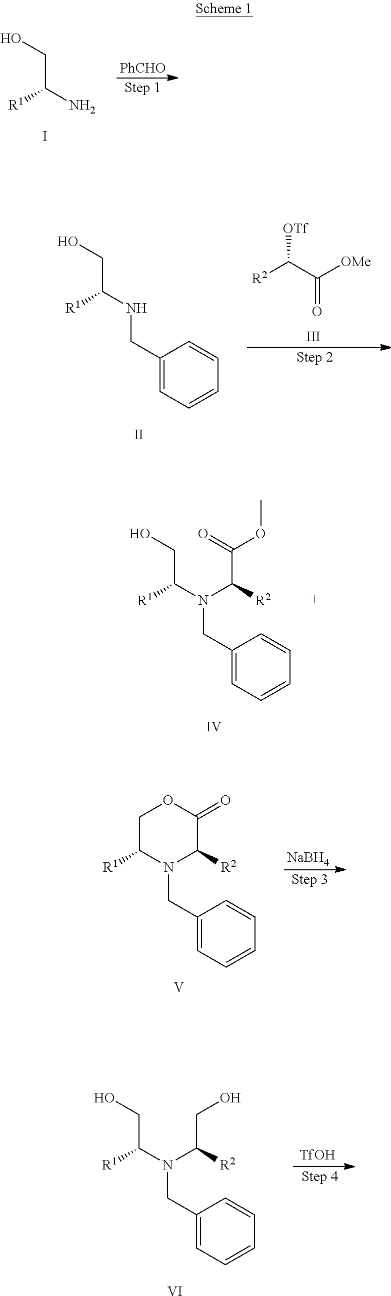

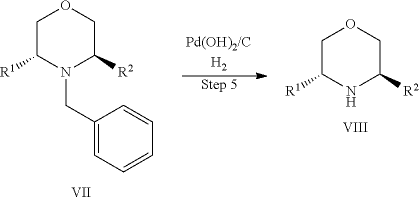

Provided herein is a method for preparing a compound of Formula (VIII)

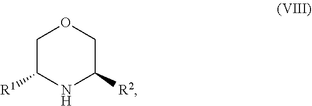

or a pharmaceutically acceptable salt, solid form, enantiomer, isotopologue, or solvate thereof,
wherein the method comprises contacting a compound of Formula (VII),

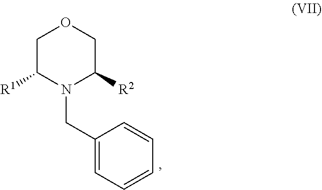

with hydrogen (H₂) in the presence of a catalyst in a suitable solvent.

In some embodiments, R¹ and R² are independently unsubstituted or substituted C₁₋₅ alkyl. In some embodiments, R¹ and R² are independently unsubstituted or substituted linear C₁₋₅ alkyl. In some embodiments, R¹ and R² are independently unsubstituted or substituted branched C₁₋₅ alkyl. In some embodiments, R¹ and R² are independently unsubstituted linear C₁₋₅ alkyl. In some embodiments, R¹ and R² are independently unsubstituted branched C₁₋₅ alkyl.

In some embodiments, R¹ and R² are independently unsubstituted or substituted C₁₋₄ alkyl. In some embodiments, R¹ and R² are independently unsubstituted or substituted linear C₁₋₄ alkyl. In some embodiments, R¹ and R² are independently unsubstituted or substituted branched C₁₋₄ alkyl. In some embodiments, R¹ and R² are independently unsubstituted linear C₁₋₅ alkyl. In some embodiments, R¹ and R² are independently unsubstituted branched C₁₋₄ alkyl.

In some embodiments, R¹ and R² are independently unsubstituted or substituted C₁₋₃ alkyl. In some embodiments, R¹ and R² are independently unsubstituted or substituted linear C₁₋₃ alkyl. In some embodiments, R¹ and R² are independently unsubstituted or substituted branched C₁₋₃ alkyl. In some embodiments, R¹ and R² are independently unsubstituted linear C₁₋₃ alkyl. In some embodiments, R¹ and R² are independently unsubstituted branched C₁₋₃ alkyl.

In some embodiments, R¹ and R² are independently unsubstituted or substituted C₁₋₂ alkyl. In some embodiments, R¹ and R² are independently unsubstituted C₁₋₂ alkyl. In some embodiments, R¹ and R² are independently substituted C₁₋₂ alkyl.

In some embodiments, R¹ and R² are independently unsubstituted or substituted C₁₋₂ alkyl. In some embodiments, R¹ and R² are independently unsubstituted C₁₋₂ alkyl. In some embodiments, R¹ and R² are independently substituted C₁₋₂ alkyl.

In some embodiments, R¹ and R² are independently unsubstituted or substituted methyl. In some embodiments, R¹ and R² are independently substituted methyl.

In some embodiments, R¹ and R² are unsubstituted methyl.

In one embodiment, the solvent is methanol, ethanol, or isopropanol.

In one embodiment, the catalyst is Pd(OH)₂/C or Pd/C.

In one embodiment, the contacting proceeds at a temperature from about 25° C. to about 55° C.

In one embodiment, the pressure of the hydrogen (H₂) is from about 1 to about 10 atm. In one embodiment, the pressure of the hydrogen (H₂) is from about 1 to about 5 atm. In one embodiment, the pressure of the hydrogen (H₂) is from about 1 to about 3 atm. In one embodiment, the pressure of the hydrogen (H₂) is about 1 atm.

In one embodiment, the compound of Formula (VII) is prepared by contacting a compound of Formula (VI),

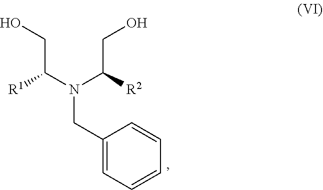

with an acid.

In one embodiment, the acid is TfOH (trifluoromethanesulfonic acid).

In one embodiment, the contacting proceeds at a temperature from about 0° C. to about 160° C. In one embodiment, the contacting proceeds at a temperature from about 20° C. to about 140° C. In one embodiment, the contacting proceeds at a temperature from about 40° C. to about 120° C. In one embodiment, the contacting proceeds at a temperature from about 50° C. to about 100° C. In one embodiment, the contacting proceeds at a temperature from about 70° C. to about 90° C. In one embodiment, the contacting proceeds at a temperature of about 80° C.

In one embodiment, the compound of Formula (VI) is prepared by contacting a mixture of a compound of Formula (IV),

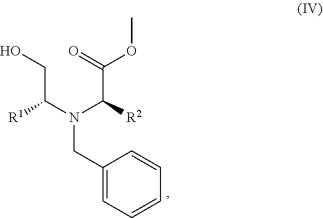

and
a compound of Formula (V),

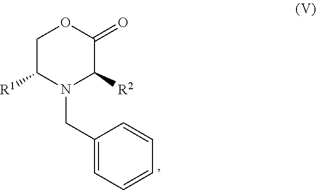

with a reducing agent.

In one embodiment, the reducing agent is NaBH₄ and the contacting proceeds in a solvent selected from the group consisting of methanol, ethanol, isopropanol and a mixture thereof at a temperature from about 15° C. to about 35° C.

In one embodiment, R¹ and R² are methyl and the compound of Formula (VI) is in a solid form at about 25° C.

In one embodiment, the compound of Formula (VI) is in a crystalline form at about 25° C.

In one embodiment, the compound of Formula (VI) has a melting point at a temperature from about 91° C. to about 93° C.

In one embodiment, the mixture of a compound of Formula (IV) and a compound of Formula (V) is prepared by contacting a compound of Formula (II),

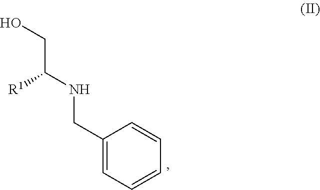

with a compound of Formula (III)

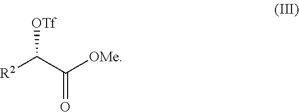

In one embodiment, the contacting proceeds at the presence of a suitable base in a suitable solvent. In one embodiment, the base is 2,6-lutidine. In one embodiment, the solvent is dichloromethane. In one embodiment, the contacting proceeds at about −10° C. to about 50° C. In one embodiment, the contacting proceeds at about 0° C. to about 40° C. In one embodiment, the contacting proceeds at about 5° C. to about 20° C.

In one embodiment, the compound of Formula (II) is prepared by contacting a compound of Formula (I),

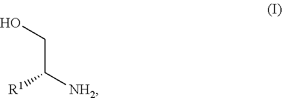

with PhCHO.

In one embodiment, the contacting proceeds at the presence of a suitable base in a suitable solvent. In one embodiment, the base is NaHCO₃. In one embodiment, the solvent is methanol. In one embodiment, the contacting proceeds at about 0° C. to about 120° C. In one embodiment, the contacting proceeds at about 20° C. to about 100° C. In one embodiment, the contacting proceeds at a temperature from about 40° C. to about 80° C. In one embodiment, the contacting proceeds at a temperature of about 62° C.

### Compounds

Provided herein are compounds of Formula (VI),

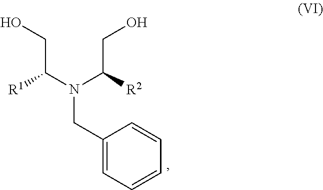

pharmaceutically acceptable salts, solid forms, enantiomer, isotopologues, and solvates thereof.

In one embodiment, a compound of Formula (VI) is a solid form. In one embodiment, a compound of Formula (VI) is a crystalline form. In one embodiment, provided herein is a solid form comprising a compound of Formula (VI). In one embodiment, provided herein is a crystalline form comprising a compound of Formula (VI).

In some embodiments, R¹ and R² are independently unsubstituted or substituted C₁₋₅ alkyl. In some embodiments, R¹ and R² are independently unsubstituted or substituted linear C₁₋₅ alkyl. In some embodiments, R¹ and R² are independently unsubstituted or substituted branched C₁₋₅ alkyl. In some embodiments, R¹ and R² are independently unsubstituted linear C₁₋₅ alkyl. In some embodiments, R¹ and R² are independently unsubstituted branched C₁₋₅ alkyl.

In some embodiments, R¹ and R² are independently unsubstituted or substituted C₁₋₄ alkyl. In some embodiments, R¹ and R² are independently unsubstituted or substituted linear C₁₋₄ alkyl. In some embodiments, R¹ and R² are independently unsubstituted or substituted branched C₁₋₄ alkyl. In some embodiments, R¹ and R² are independently unsubstituted linear C₁₋₅ alkyl. In some embodiments, R¹ and R² are independently unsubstituted branched C₁₋₄ alkyl.

In some embodiments, R¹ and R² are independently unsubstituted or substituted C₁₋₃ alkyl. In some embodiments, R¹ and R² are independently unsubstituted or substituted linear C₁₋₃ alkyl. In some embodiments, R¹ and R² are independently unsubstituted or substituted branched C₁₋₃ alkyl. In some embodiments, R¹ and R² are independently unsubstituted linear C₁₋₃ alkyl. In some embodiments, R¹ and R² are independently unsubstituted branched C₁₋₃ alkyl.

In some embodiments, R¹ and R² are independently unsubstituted or substituted C₁₋₂ alkyl. In some embodiments, R¹ and R² are independently unsubstituted C₁₋₂ alkyl. In some embodiments, R¹ and R² are independently substituted C₁₋₂ alkyl.

In some embodiments, R¹ and R² are independently unsubstituted or substituted C₁₋₂ alkyl. In some embodiments, R¹ and R² are independently unsubstituted C₁₋₂ alkyl. In some embodiments, R¹ and R² are independently substituted C₁₋₂ alkyl.

In some embodiments, R¹ and R² are independently unsubstituted or substituted methyl. In some embodiments, R¹ and R² are independently substituted methyl.

### Compound 6

Provided herein is Compound 6 having the name of (2R,2′R)-2,2′-(benzylazanediyl)bis(propan-1-ol) with the following structure:

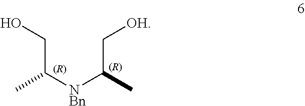

In one embodiment, Compound 6 is a solid form. In one embodiment, Compound 6 is a crystalline form. In one embodiment, provided herein is a solid form comprising Compound 6.

### Form A

In certain embodiments, provided herein is Form A of Compound 6 only by way of example, and without limitation.

In one embodiment, Form A is a solid form of Compound 6. In one embodiment, Form A is an anhydrous solid form of Compound 6. In another embodiment, Form A is crystalline. In another embodiment, Form A has an X-ray powder diffraction pattern comprising peaks at approximately 22.5, 27.1, and 27.3° 2θ. In another embodiment, Form A has a melting point at a temperature from about 91° C. to about 93° C.

In certain embodiments, Form A provided herein is obtained by recrystallization experiments and anti-solvent recrystallization experiments. In certain embodiments, Form A is obtained from certain solvent systems including toluene, heptane, water, methanol and a mixture thereof.

In one embodiment, a method of preparing Form A comprises the steps of 1) mixing Compound 6 with a solvent (e.g., toluene) mixture containing n-heptane (e.g., at least about 75% by volume of n-heptane); 2) stirring at a temperature (e.g., from about 0° C. to about 10° C., such as about 5° C.) for a period of time (e.g., from about 1 hour to about 6 hours, such as about 3 hours); and 3) collecting solids and optionally drying.

In one embodiment, a method of preparing Form A comprises the steps of 1) mixing Compound 6 with a solvent (e.g., methanol) mixture containing n-heptane; 2) heating to a temperature (e.g., from between about 0° C. to about 75° C., such as about 50° C.) for a period of time (e.g., from about 1 hour to about 6 hours, such as about 3 hours); 3) cooling to a second temperature (e.g., from between about 0° C. to about 50° C., such as about 25° C.); and 4) collecting solids and optionally drying.

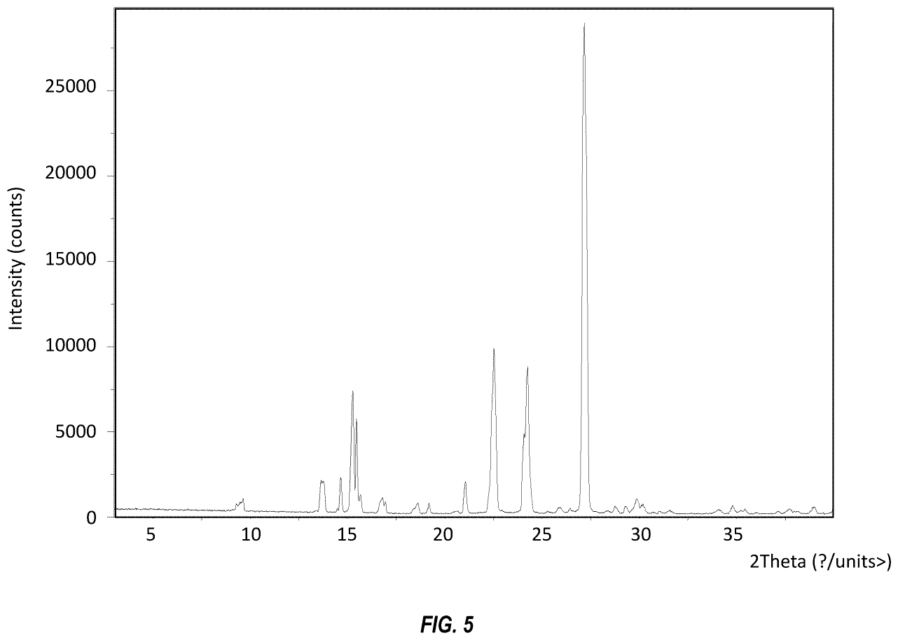

In certain embodiments, a solid form provided herein, e.g., Form A of Compound 6, is substantially crystalline, as indicated by, e.g., X-ray powder diffraction measurements. In one embodiment, Form A has an X-ray powder diffraction pattern substantially as shown in FIG. 5. In one embodiment, Form A has one or more characteristic X-ray powder diffraction peaks at approximately 9.3, 9.6, 13.6, 13.8, 14.7, 15.3, 15.5, 15.7, 16.7, 16.8, 17.0, 18.6, 19.2, 20.6, 21.0, 22.4, 22.5, 24.0, 24.2, 25.3, 25.9, 26.4, 27.1, 27.3, 28.4, 28.7, 29.3, 29.9, 30.1, 31.0, 31.5, 34.1, 34.8, 35.4, 37.1, 37.7, 38.2, or 39.0° 2θ as depicted in FIG. 5. In a specific embodiment, Form A has one, two, three, four, five, six, seven, eight, nine, ten, eleven, or twelve characteristic X-ray powder diffraction peaks at approximately 13.6, 13.8, 14.7, 15.3, 15.5, 21.0, 22.4, 22.5, 24.0, 24.2, 27.1, or 27.3° 2θ. In another embodiment, Form A has one, two, three, four, five, six, seven, eight, or nine characteristic X-ray powder diffraction peaks at approximately 14.7, 15.3, 15.5, 22.4, 22.5, 24.0, 24.2, 27.1, or 27.3° 2θ. In another embodiment, Form A has one, two, three, four, five, or six characteristic X-ray powder diffraction peaks at approximately 15.3, 22.4, 22.5, 24.2, 27.1, or 27.3° 2θ. In another embodiment, Form A has one, two, or three characteristic X-ray powder diffraction peaks at approximately 22.5, 27.1, or 27.3° 2θ. In another embodiment, Form A has one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, thirty-seven, or thirty-eight characteristic X-ray powder diffraction peaks as set forth in Table 1.

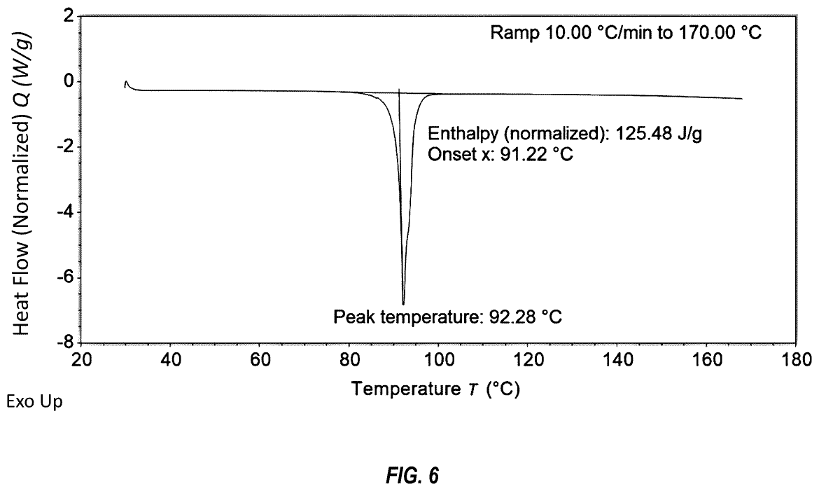

In one embodiment, provided herein is Form A having a DSC thermogram substantially as depicted in FIG. 6 comprising an endothermic event with an onset temperature of about 91° C. and a peak temperature of about 92° C. when heated from approximately 25° C. to approximately 150° C.

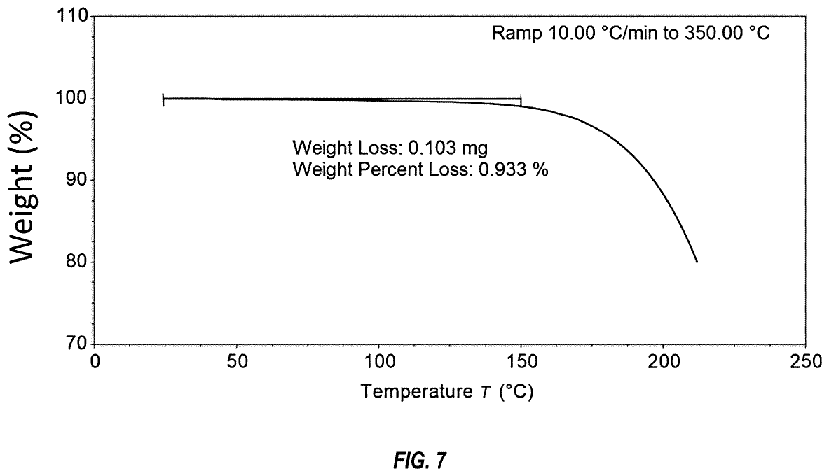

In one embodiment, provided herein is Form A having a TGA thermograph corresponding substantially to the representative TGA thermogram as depicted in FIG. 7. In certain embodiments, the crystalline form exhibits a TGA thermogram comprising a total mass loss of approximately 1% of the total mass of the sample between approximately 30° C. and approximately 150° C. when heated from approximately 20° C. to approximately 300° C. Thus, in certain embodiments, the crystalline form loses from about 0.1% to about 5%, for example, about 0.5% or about 3%, of its total mass when heated from about ambient temperature to about 150° C.

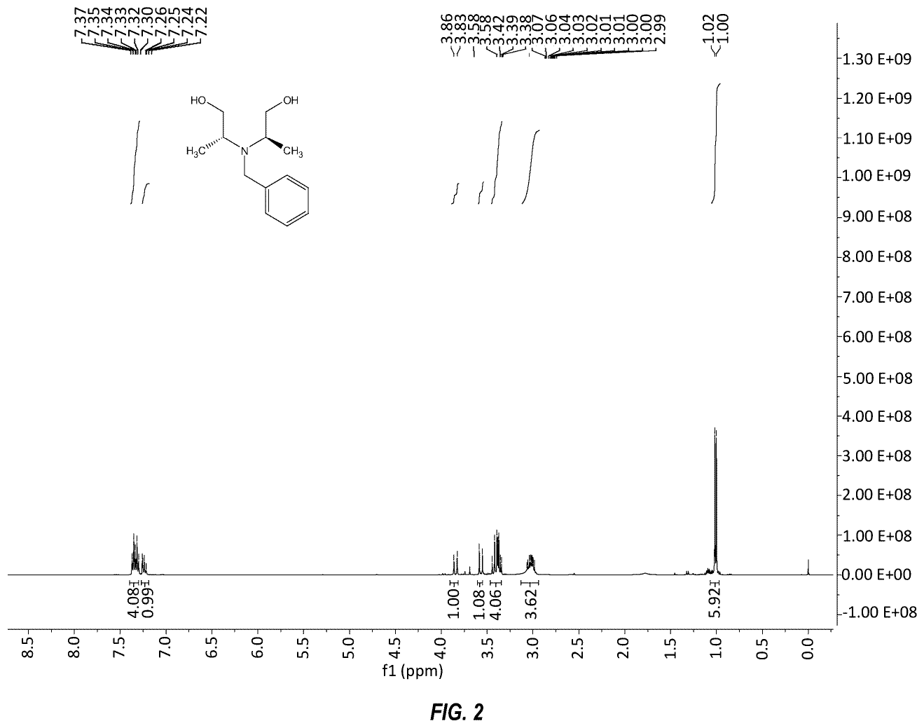

In one embodiment, provided herein is Form A having a ¹H NMR spectrum substantially as depicted in FIG. 2.

In still another embodiment, Form A is substantially pure. In certain embodiments, the substantially pure Form A is substantially free of other solid forms, e.g., amorphous solid. In certain embodiments, the purity of the substantially pure Form A is no less than about 95%, no less than about 96%, no less than about 97%, no less than about 98%, no less than about 98.5%, no less than about 99%, no less than about 99.5%, or no less than about 99.8%.

It should be noted that if there is a discrepancy between a depicted structure and a name given to that structure, the depicted structure is to be accorded more weight.

### EXAMPLES

### Abbreviations

The following Examples are presented by way of illustration, not limitation. The following abbreviations are used in descriptions and examples:
DCM: Dichloromethane
DIPEA: N,N-Diisopropylethylamine
MeOH: Methanol
MTBE: tert-Butyl methyl ether
NMP: N-Methyl-2-pyrrolidone
NMR: Nuclear magnetic resonance
Tf: Triflate or trifluoromethanesulfonyl
Tf₂O: Trifluoromethanesulfonic anhydride

### Synthetic Examples

The following synthetic examples, presented by way of illustration and not limitation, show methods for the preparation of the compound provided herein. Chemdraw 18.0.0.231 published 2018 (Cambridgesoft, Perkin Elmer, Waltham, MA) was used to draw the chemical structures and generate names for chemical structures.

### Example 1: Synthesis of (3R,5R)-3,5-dimethylmorpholine hydrochloride

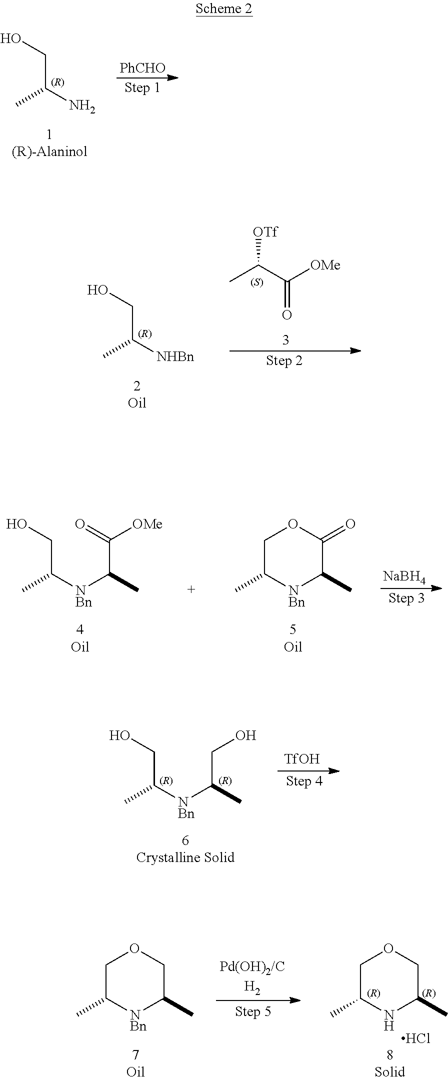

(R)-2-(benzylamino)propan-1-ol (2): To a 5 L three-necked round-bottomed flask were added 2.0 L of MeOH (10 V), 200.0 g of PhCHO (1.0 eq), 162.8 g (R)-alaninol (1.15 eq), and 237.6 g of NaHCO₃ (1.5 eq) with stirring at 25-35° C. to form a suspension solution. After heating to reflux for 4 hours with the inner temperature at 62° C., the batch was cooled to 20-25° C., and filtered to remove undissolved solids, rinsing the filter cake with MeOH (1 V).

The filtrate was transferred to a dry and clean 5 L three-necked round-bottomed flask. To the flask was added 39.2 g of NaBH₄ (0.55 eq) in portions slowly with controlling the inner temperature at 25° C.˜35° C. (Cautions: bubbling and exothermic). The reaction mixture was stirred at 10-30° C. for 16 hours.

Upon completion of the reaction, as indicated by HPLC, the batch was quenched with aqueous NH₄Cl (11.5 g in 11.5 mL of water). The batch was stirred for 0.5 hour and was concentrated to ˜700 mL (˜3 V) under vacuum. The reaction mixture was swapped with 500 mL of MTBE to (˜2 V). 1.2 L of MTBE was added to the residue and the reaction mixture was stirred for 0.5 hour at 20-35° C. The batch was filtered through a celite pad and rinsed with 1.0 L of MTBE. The filtrate was concentrated to dryness under vacuum.

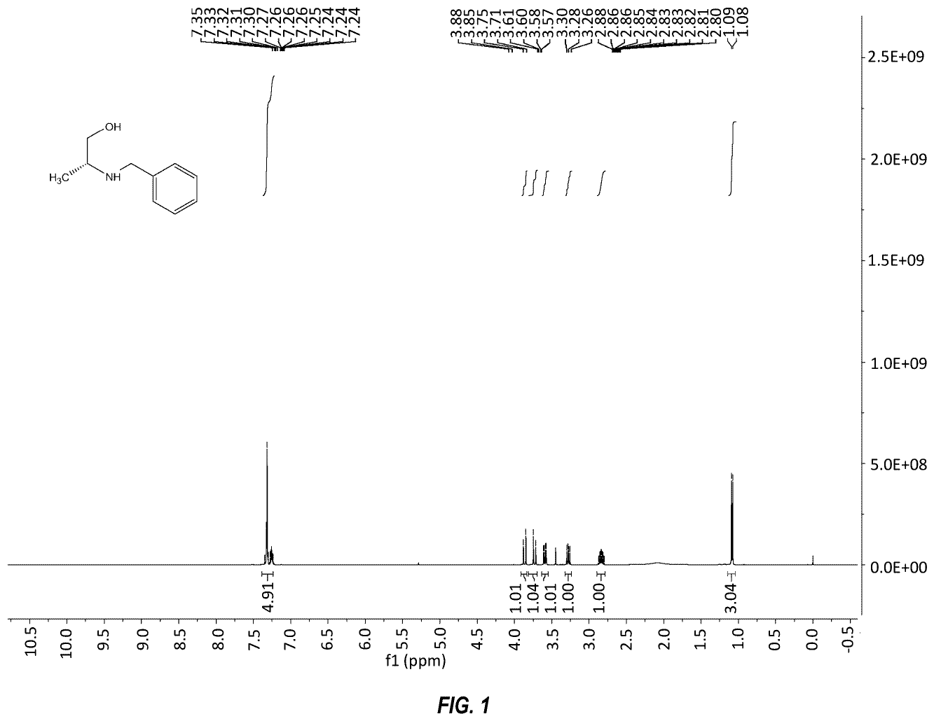

295.2 g of (R)-2-(benzylamino)propan-1-ol (2) was obtained as crude oil with 98.2% HPLC purity and an ¹H NMR spectrum as provided in FIG. 1.

### Methyl N-benzyl-N-((R)-1-hydroxypropan-2-yl)-D-alaninate (4) and (3R,5R)-4-benzyl-3,5-dimethylmorpholin-2-one (5)

To a 5 L three-necked round-bottomed flask were added 1.8 L of DCM, 216.3 g of methyl (S)-(−)-lactate (1.25 eq) and 240.3 g of 2,6-lutidine (1.35 eq) with stirring at 10 to 30° C. The flask was degassed and re-filled with nitrogen for three times. The batch was cooled to −40 to −20° C. 609.9 g of Tf₂O (1.3 eq) was added dropwise to the reaction mixture over 1.5 hours with temperature controlling at −35 to −25° C. The reaction mixture was then warmed to −5 to 0° C. and stirred for 1 hour. 600 mL of ice-water was added into the reaction mixture and the reaction mixture was stirred for 0.5 hour at 0° C. The organic phase was separated at 0° C. and transferred to a 5 L three-necked round-bottomed flask. To the flask was added 430.1 g of DIPEA (2.2 eq) at 0° C. and then a solution of 295.2 g of crude (R)-2-(benzylamino)propan-1-ol (2) (1.0 eq) from the above procedure in 600 mL of DCM over 1 hour at 0° C. The reaction mixture was warmed to 10 to 15° C. and stirred for 16 hours. The reaction mixture was washed with water (1 L×3).

The DCM phases were separated, combined, and concentrated under vacuum to dryness. To the reaction mixture was added 1.2 L of MTBE to dissolve the reaction mixture. The reaction mixture was stirred at 10 to 20° C. for 0.5 hour and filtered. The cake was rinsed with MTBE (400 mL). The organic phases were combined and concentrated to dryness under vacuum to obtain 463.2 g of crude product as a mixture of methyl N-benzyl-N-((R)-1-hydroxypropan-2-yl)-D-alaninate (4) (55.1%, a/a) and (3R,5R)-4-benzyl-3,5-dimethylmorpholin-2-one (5) (34.1%, a/a) (89.2%, a/a).

### (2R,2′R)-2,2′-(benzylazanediyl)bis(propan-1-ol) (6)

To a 5 L three-necked round-bottomed flask were added 2.3 L of MeOH, and 463.2 g of a mixture of methyl N-benzyl-N-((R)-1-hydroxypropan-2-yl)-D-alaninate (4) and (3R,5R)-4-benzyl-3,5-dimethylmorpholin-2-one (5) (1.0 eq). To the reaction mixture was added 146.6 g of NaBH₄(2.0 eq) in portions at 20-30° C. The reaction mixture was stirred for another 16 hours.

Aqueous NH₄Cl (40 g in 200 mL of H₂O) was added into the mixture to quench the reaction. The reaction mixture was concentrated under vacuum to remove most of MeOH, swapped with 1 L of n-heptane, and then triturated with 1.0 L of water and 2.0 L n-heptane.

The reaction mixture was filtered. The cake was rinsed with n-heptane (500 mL), collected, and dried at 50° C. under vacuum.

257.0 g of (2R,2′R)-2,2′-(benzylazanediyl)bis(propan-1-ol) (6) was obtained as yellow solid in yield ˜61% for 3 steps. The obtained solid has 97.3% chemical purity with d.r.: 99.3:0.7 and gave the ¹H NMR provided in FIG. 2.

It is surprising and unexpected that (2R,2′R)-2,2′-(benzylazanediyl)bis(propan-1-01) (6) is a crystalline solid, which was confirmed as Form A. It is further surprising and unexpected that (2R,2′R)-2,2′-(benzylazanediyl)bis(propan-1-ol) (6) crystallized out of the reaction mixture selectively under the disclosed conditions. This method effectively removed the undesirable diastereomers and other by-products. Furthermore, this method generated the desirable diastereomer with high d.r.

### Procedure to Improve d.r. of (2R,2′R)-2,2′-(benzylazanediyl)bis(propan-1-ol) (6)

To a 1 L three-necked round-bottomed flask were added 127 mL (2 V) of toluene, 635 mL of n-heptane (10 V), and 63.5 g of (2R,2′R)-2,2′-(benzylazanediyl)bis(propan-1-ol) (6) (1.0 eq, d.r.: 99.4:0.6). The reaction mixture was stirred at 100° C. to form a clear solution and then stirred at 100° C. for another hour. The reaction mixture was cooled down to 5° C. over 2 hours and stirred for another 1 hour. The reaction mixture was filtered. The collected solid was rinsed with 127 mL of (2V) n-heptane and dried at 50° C. under vacuum.

59.5 g of (2R,2′R)-2,2′-(benzylazanediyl)bis(propan-1-ol) (6) was obtained as yellow solid in yield ˜93%. The obtained solid, which was also confirmed as Form A, has 98.4% chemical purity with >98.5:1.5 d.r.

### (3R,5R)-4-benzyl-3,5-dimethylmorpholine (7)

To a 500 mL clean and dry three-necked flask were added 180 mL of TfOH (3.0 V), and 58.5 g of (2R,2′R)-2,2′-(benzylazanediyl)bis(propan-1-ol) (6) (1.0 eq, dr.: >98.5:1.5) in portions with stirring at 20 to 35° C. The reaction mixture was stirred at 80° C. for 16 hours and then cooled down to 20˜30° C. To the reaction mixture was added dropwise to the 20% aqueous NaOH (500 g) slowly with stirring at 0-10° C. (make sure pH >12). The reaction mixture was extracted with MTBE (250 mL×2). The MTBE phases were combined and filtered. The filtrate was concentrated under vacuum.

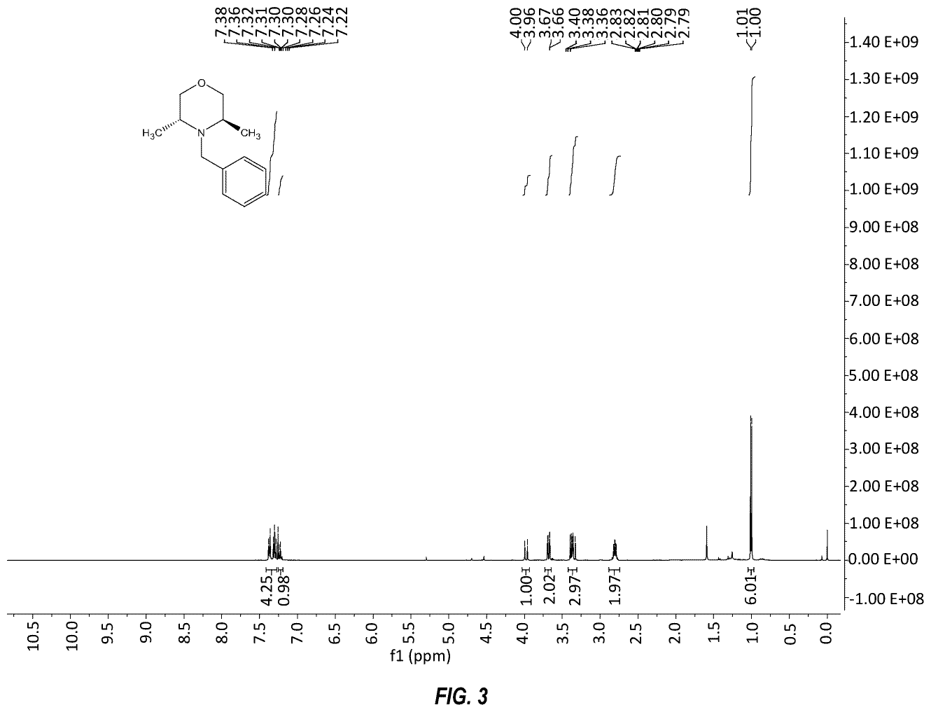

51.2 g of (3R,5R)-4-benzyl-3,5-dimethylmorpholine (7) was obtained as oil in 89% yield and 99.6% purity, >99.0% e.e., and >98.5:1.5 d.r. and gave material exhibiting the ¹H NMR spectrum provided in FIG. 3.

### (3R,5R)-3,5-dimethylmorpholine hydrochloride salt (8)

To a 500 mL clean and dry three-necked flask were added 255 mL of MeOH (5 V), 51.0 g of (3R,5R)-4-benzyl-3,5-dimethylmorpholine (7) (1.0 eq), and 3.6 g of Pd(OH)₂/C (7% wt vs. Compound 7, 0.5 mol % Pd, moisture content 65%, from Kaili Catalyst Co.). The autoclave was de-gassed and refilled with hydrogen three times. The reaction mixture was stirred under 1 atm of hydrogen at 35 to 40° C. for 16 hours. The reaction mixture was filtered under nitrogen. The collected solid was rinsed with 100 mL of MeOH. To the filtrate was added 100 mL of HCl (4M in 1,4-dioxane) to reach pH<3. The reaction mixture was stirred for 1 hour, concentrated under vacuum and then swapped with isopropanol (150 mL×2). The reaction mixture was stirred with 51 mL of isopropanol (1 V) and 102 mL of n-heptane (2 V) for 16 hours. The reaction mixture was filtered. The collected solid was rinsed with 51 mL of n-heptane and dried in an oven under vacuum at 50° C. for 16 hours.

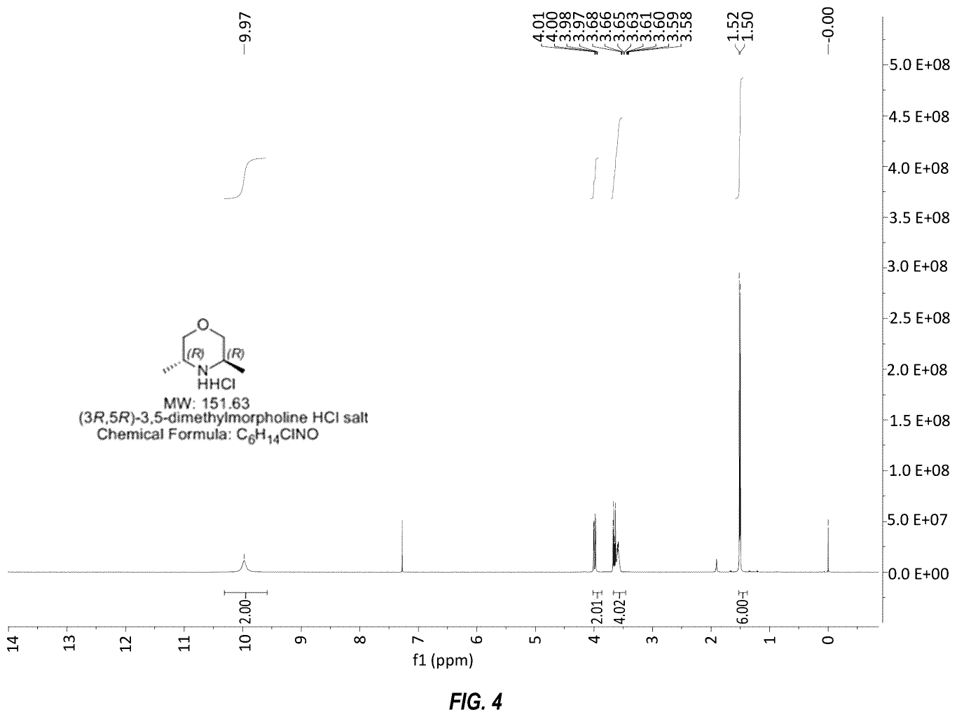

32.4 g of (3R,5R)-3,5-dimethylmorpholine hydrochloride salt (8) was obtained as solid in 86% yield. The obtained solid showed a ¹H NMR spectrum as provided in FIG. 4.

### Form A

Form A is an anhydrous crystalline solid form of Compound 6. This form was obtained from recrystallization from a mixture of toluene and n-heptane, or a mixture of methanol and n-heptane.

Form A has a crystalline XRPD pattern as shown in FIG. 5. TGA and DSC thermograms of Form A are shown in FIG. 6 and FIG. 7, respectively. The DSC thermogram showed only one major event with an onset temperature of 91.2° C. and a peak temperature of 92.3° C., corresponding to melt/decomposition. TGA weight loss of 1% was observed up to 150° C.

FIG. 5 provides an XRPD pattern of Form A. A list of X-Ray Diffraction Peaks for Form A is provided below in Table 1.

TABLE 1
X-Ray Diffraction Peaks for Form A

| Two-theta angle (°) | d Space (Å) | Relative Intensity (%) |
|---|---|---|
| 9.2975 | 9.51220 | 1.51 |
| 9.6396 | 9.17542 | 2.90 |
| 13.6186 | 6.50221 | 7.06 |
| 13.8062 | 6.41429 | 6.64 |
| 14.6517 | 6.04600 | 7.99 |
| 15.2771 | 5.79985 | 28.53 |
| 15.4733 | 5.72678 | 21.66 |
| 15.6831 | 5.65061 | 4.01 |
| 16.6494 | 5.32478 | 2.27 |
| 16.7895 | 5.28067 | 3.53 |
| 16.9543 | 5.22970 | 2.61 |
| 18.6213 | 4.76514 | 2.41 |
| 19.1938 | 4.62426 | 2.33 |
| 20.6281 | 4.30587 | 0.53 |
| 21.0429 | 4.22192 | 7.24 |
| 22.4030 | 3.96858 | 22.86 |
| 22.5447 | 3.94396 | 37.83 |
| 24.0122 | 3.70615 | 13.85 |
| 24.2412 | 3.67165 | 34.10 |
| 25.2864 | 3.52221 | 0.32 |
| 25.8939 | 3.44093 | 1.06 |
| 26.4189 | 3.37374 | 0.67 |
| 27.0933 | 3.29127 | 100.00 |
| 27.3037 | 3.26638 | 68.79 |
| 28.3526 | 3.14788 | 0.38 |
| 28.7294 | 3.10745 | 1.41 |
| 29.2635 | 3.05194 | 1.29 |
| 29.8656 | 2.99177 | 3.04 |
| 30.1571 | 2.96351 | 1.75 |
| 31.0181 | 2.88318 | 0.47 |
| 31.5366 | 2.83696 | 0.66 |
| 34.1130 | 2.62836 | 0.80 |
| 34.8040 | 2.57775 | 1.69 |
| 35.4315 | 2.53352 | 0.92 |
| 37.1286 | 2.42152 | 0.53 |
| 37.7086 | 2.38560 | 0.95 |
| 38.1533 | 2.35881 | 0.42 |
| 39.0344 | 2.30757 | 1.42 |

The conditions for measuring the XRPD pattern are provided below in Table 2.

TABLE 2
XRPD measurement conditions

| | |
|---|---|
| Scan Axis | Gonio |
| Start Position [°2θ] | 3.0131 |
| End Position [°2θ] | 39.9851 |
| Step Size [°2θ] | 0.0260 |
| Scan Step Time [s] | 36.4650 |
| Scan Type | Continuous |
| PSD Mode | Scanning |
| PSD Length [°2θ] | 3.35 |
| Offset [°2θ] | 0.0000 |
| Divergence Slit Type | Fixed |
| Divergence Slit Size [°] | 0.1799 |
| Specimen Length [mm] | 10.00 |
| Measurement Temperature [° C.] | 25.00 |
| Anode Material | Cu |
| Intended Wavelength Type | K-Alpha |
| K-Alpha1 [Å] | 1.54060 |
| K-Alpha2 [Å] | 1.54443 |
| K-Beta [Å] | 1.39225 |
| K-A2/K-A1 Ratio | 0.50000 |
| Generator Settings | 40 mA, 45 kV |
| Diffractometer Type | 0000000011254373 |
| Diffractometer Number | 0 |
| Goniometer Radius [mm] | 240.00 |
| Dist. Focus-Diverg. Slit [mm] | 60.50 |
| Incident Beam Monochromator | No |
| Spinning | Yes |

A number of references have been cited, the disclosures of which are incorporated herein by reference in their entirety.

1. A compound of Formula (VI), 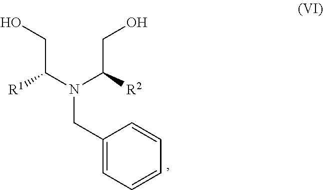
or a pharmaceutically acceptable salt, solid form, enantiomer, isotopologue, or solvate thereof,
wherein
R¹ and R² are independently unsubstituted or substituted C₁₋₅ alkyl.

2. The compound of claim 1, wherein R¹ and R² are independently unsubstituted or substituted C₁₋₄ alkyl.

3. The compound of claim 1, wherein R¹ and R² are independently unsubstituted linear or branched C₁₋₄ alkyl.

4. (canceled)

5. The compound of claim 1, wherein R¹ and R² are methyl.

6. A solid form comprising the compound of claim 1.

7. A crystal form comprising the compound of claim 5, wherein the crystal form has an X-ray powder diffraction pattern comprising one, two or three peaks at 22.5, 27.1, or 27.3±0.2° 2θ.

8. The crystal form of claim 7, wherein the X-ray powder diffraction pattern further comprises one, two or three peaks at 15.3, 22.4, or 24.2±0.2° 2θ.

9. The crystal form of claim 7, wherein the crystal form has a melting point at a temperature from about 91° C. to about 93° C.

10. The crystal form of claim 7, wherein the crystal form is anhydrous.

11. A method for preparing a compound of Formula (VIII): 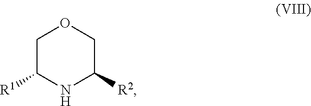
or a pharmaceutically acceptable salt, solid form, enantiomer, isotopologue, or solvate thereof,
wherein the method comprises contacting a compound of Formula (VII): 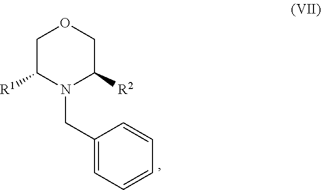
with hydrogen (H₂) in the presence of a catalyst in a solvent, wherein
R¹ and R² are independently unsubstituted or substituted C₁₋₅ alkyl.

12. The method of claim 11, wherein the solvent is methanol, ethanol, or isopropanol.

13. The method of claim 11, wherein the catalyst is Pd(OH)₂/C or Pd/C.

14. The method of claim 11, wherein the pressure of the hydrogen (H₂) is about 1 to about 10 atm and the contacting proceeds at a temperature from about 25° C. to about 55° C.

15. The method of claim 14, wherein the pressure of the hydrogen (H₂) is about 1 to about 5 atm.

16. The method of claim 14, wherein the pressure of the hydrogen (H₂) is about 1 to about 3 atm.

17. The method of claim 11, wherein the compound of Formula (VII) is prepared by contacting a compound of Formula (VI): 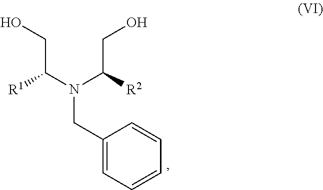
with an acid.

18. The method of claim 17, wherein the acid is TfOH and the contacting proceeds at a temperature from about 20° C. to about 140° C.

19. The method of claim 17, wherein the compound of Formula (VI) is prepared by contacting a mixture of a compound of Formula (IV): 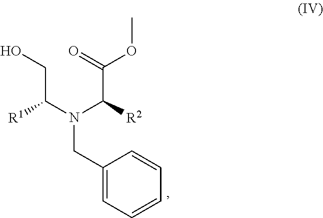
and
a compound of Formula (V): 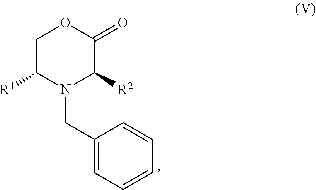
with a reducing agent.

20. The method of claim 19, wherein the reducing agent is NaBH₄ and the contacting proceeds in a solvent selected from the group consisting of methanol, ethanol, isopropanol, and a mixture thereof at a temperature from about 15° C. to about 35° C.

21. The method of claim 17, wherein the compound of Formula (VI) is a solid form.

22. The method of claim 17, wherein R¹ and R² are methyl.

23. The method of claim 22, wherein the compound of Formula (VI) is a crystalline form comprising the compound of Formula (VI), wherein the crystal form has an X-ray powder diffraction pattern comprising one, two or three peaks at 22.5, 27.1, or 27.3±0.2° 2θ.

24. The method of claim 23, wherein the X-ray powder diffraction pattern further comprises one, two, or three peaks at 15.3, 22.4, or 24.2±0.2° 2θ.

25. The method of claim 23, wherein the crystal form has a melting point at a temperature from about 91° C. to about 93° C.

26. The method of claim 23, wherein the crystal form is anhydrous.

27. The method of claim 19, wherein the mixture of a compound of Formula (IV) and a compound of Formula (V) is prepared by contacting a compound of Formula (II): 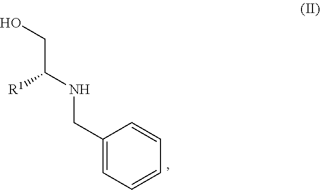
with a compound of Formula (III): 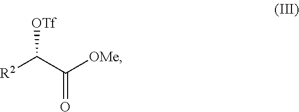
in the presence of a suitable base in a suitable solvent.

28. The method of claim 27, wherein the suitable base is 2,6-lutidine; the suitable solvent is dichloromethane; and the contacting proceeds at a temperature from about 0° C. to about 40° C.

29. The method of claim 27, wherein the compound of Formula (II) is prepared by contacting a compound of Formula (I), 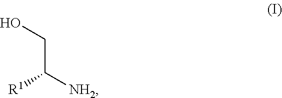
with PhCHO in the presence of a suitable base in a suitable solvent.

30. The method of claim 29, wherein the suitable base is NaHCO₃; the suitable solvent is methanol; and the contacting proceeds at a temperature from about 20° C. to about 40° C.
